(19) United States
(12) Patent Application Publication
(10) Pub. No.: US 2026/0355433 A1
(43) Pub. Date: Nov. 12, 2026

(54) Portable Alcohol Vaporizer

(71) Applicant: Kimberley Lynn Bogema, Scarborough (CA)

(72) Inventor: Kimberley Lynn Bogema, Scarborough (CA)

(21) Appl. No.: 19/204,669

(22) Filed: May 12, 2025

(51) Int. Cl. A61M 15/00; H05B 3/00

(52) U.S. Cl. CPC A61M 15/002; A61M 15/0021; H05B 3/0014; A61M 2202/02; A61M 2205/27; A61M 2205/3673; A61M 2205/8206; A61M 2205/8237; H05B 2203/021

(57) ABSTRACT

A portable alcohol vaporizer is provided. The portable alcohol vaporizer includes a housing with a handle body and a tip that forms a pen-shaped structure when secured together via a fastener. The device includes a liquid reservoir that can hold a concentrated alcohol compound, a heating element disposed in a chamber for vaporizing the compound into alcohol vapor, and a vapor tube delivering the vaporized alcohol to a mouthpiece. A printed circuit board assembly (PCBA) controls the vaporization process and is operatively connected to a rechargeable battery housed within the handle body. The portable alcohol vaporizer includes a refillable or interchangeable liquid reservoir, a wick or pump for controlled alcohol delivery to the heating element, and user interfaces such as a button or sensor for activation. The device provides a discreet, efficient, and user-controlled method of alcohol consumption.

### BACKGROUND OF THE INVENTION

The present invention relates to alcohol vaporization devices, and more specifically, to a portable alcohol vaporizer designed for inhaling vaporized alcohol compounds. The device provides a compact, efficient, and controlled method for alcohol consumption, addressing challenges associated with traditional methods of alcohol intake.

Traditional methods of alcohol consumption involve the ingestion of liquid alcohol, which is metabolized through the digestive system. These methods have several drawbacks, including a delay in the onset of alcohol's effects, the ingestion of empty calories, and limitations in settings where alcohol use is restricted or monitored. Additionally, individuals with medical conditions affecting their digestive systems may struggle to consume alcohol safely or effectively.

Existing methods for alcohol inhalation typically involve rudimentary or unsafe practices, such as heating alcohol in an open container or using an open flame. These approaches lack portability, are prone to safety hazards such as burns or unregulated vaporization, and fail to provide precise control over alcohol delivery.

While devices for inhaling other compounds, such as nicotine or cannabis, have become commercially available and widely used, there is no equivalent portable, safe, and efficient solution for alcohol vaporization. Such a device would provide a novel approach to alcohol consumption while addressing the safety, usability, and portability issues found in existing practices.

The present invention addresses these shortcomings by providing a portable alcohol vaporizer that is compact, pen-shaped, and easy to use. The device features a liquid reservoir for a concentrated alcohol compound, a heating element to vaporize the compound, and a mouthpiece for inhaling the vaporized alcohol. It incorporates advanced safety features such as a printed circuit board assembly (PCBA) for controlled operation, a refillable or interchangeable reservoir, and user interfaces such as sensors or buttons to ensure user control.

In light of the devices disclosed in the known art, it is submitted that the present invention substantially diverges in design elements and methods from the known art and consequently it is clear that there is a need in the art for an improvement for a portable alcohol vaporization device. In this regard the instant invention substantially fulfills these needs.

### SUMMARY OF THE INVENTION

In view of the limitations and safety concerns associated with traditional alcohol consumption methods and prior vaporization devices, the present invention provides a portable alcohol vaporizer that enables the controlled vaporization and inhalation of concentrated alcohol compounds. The device offers a discreet, efficient, and user-friendly alternative for alcohol consumption, addressing challenges of portability, safety, and user control.

The portable alcohol vaporizer comprises a housing with a handle body and a tip, forming a pen-shaped structure when secured via a fastener. The device includes a liquid reservoir configured to hold a concentrated alcohol compound, a heating element disposed within a chamber for vaporizing the alcohol compound, and a vapor tube for delivering vaporized alcohol to a mouthpiece.

It is an objective of the present invention to include a rechargeable battery housed within the handle body and a printed circuit board assembly (PCBA) configured to control the vaporization process. This provides a metered dosage of alcohol to the user.

It is an objective of the present invention to include safety and usability features, such as a wick or pump to regulate the transfer of alcohol to the heating element, a refillable or interchangeable liquid reservoir, and user interfaces such as actuatable buttons or mouthpiece sensors for controlled activation.

It is an objective of the present invention to provide a pen-shaped, compact, and ergonomically configured housing that ensures portability, concealment, and ease of use in various environments.

It is an objective of the present invention to provide a harm-reduction solution for individuals with medical conditions affecting the digestive system. The inhalation of alcohol bypasses traditional metabolic pathway which is a benefit to individuals with digestive conditions.

It is an objective of the present invention to provide a portable alcohol vaporizer that has all of the advantages of the known art and none of the disadvantages.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of one embodiment of the portable alcohol vaporizer, showing the assembled housing with the handle body and tip forming a pen-shaped structure.

FIG. 2 shows a semi-cross-sectional view of the portable alcohol vaporizer, taken along lines 2-2 of FIG. 1.

FIG. 3 shows a block diagram of one embodiment of the assembled portable alcohol vaporizer.

### DETAILED DESCRIPTION OF THE INVENTION

Reference is made herein to the attached drawings. Like reference numerals are used throughout the drawings to depict like or similar elements of the portable alcohol vaporizer. For the purpose of presenting a clear description of the present invention, the preferred embodiment will be discussed as used for providing a compact, efficient, and controlled method for alcohol consumption. The figures are intended for representative purposes only and should not be considered limiting in any respect.

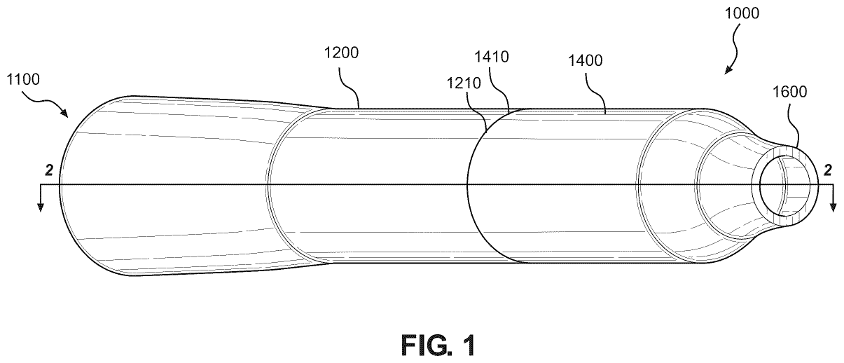

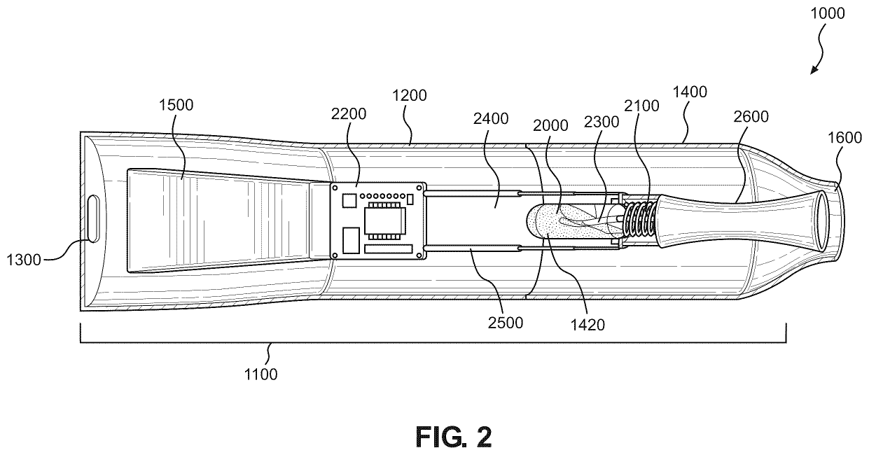

Referring now to FIG. 1, there is shown a perspective view of one embodiment of the portable alcohol vaporizer, showing the assembled housing with the handle body and tip forming a pen-shaped structure. The portable alcohol vaporizer 1000 facilitates the controlled vaporization and inhalation of a concentrated alcohol compound, providing a user-friendly and efficient alternative to traditional alcohol consumption methods. The portable alcohol vaporizer 1000 includes a housing 1100 comprising a handle body 1200 and a tip 1400 that, when secured together, form a pen-shaped structure. The housing 1000 is portable and discrete. In the shown embodiment, the housing 1100 forms a continuous exterior surface between the handle body 1200 and the tip 1400. The handle body 1200 and the tip 1400 are removably connected via mating threaded fasteners 1210, 1410 (shown in FIG. 2). In this manner, the vaporizer 1000 is configured to be disassembled for maintenance, refilling, or replacement of components. However, in alternate embodiments, the handle body and tip are fastened via any suitable fastening mechanism.

In the shown embodiment, the handle body 1200 houses a rechargeable battery 1500 configured to power the vaporizer 1000, as well as a charging port 1300 for recharging the battery 1500. The tip 1400 contains a liquid reservoir 1420 configured to hold a concentrated alcohol compound 2000. In one embodiment, the liquid reservoir 1420 is refillable via a refill port, enabling users to replenish the compound as needed. In another embodiment, it is an interchangeable cartridge that can be replaced with a new reservoir, allowing users to customize or replenish the device based on their preferences.

In some embodiments, the concentrated alcohol compound includes flavors. In other embodiments, the concentrated alcohol compound includes infusions with cannabis, nicotine, and the like. In some embodiment, the reservoir is configured to housing 10 ml of the concentrated alcohol compound. In another embodiment, the reservoir is configured to house between 2 ml-100 ml of the concentrated alcohol compound.

In the shown embodiment, the mouthpiece 1600 is positioned at the distal end of the tip 1400 and is configured to provide a pathway for inhaling vaporized alcohol. The vaporizer 1000 also includes a vapor tube (shown in FIG. 2) that connects the chamber containing the heating element to the mouthpiece 1600. This configuration ensures that the alcohol vapor is efficiently delivered to the user while minimizing condensation or loss during transport. The mouthpiece is tapered, decreasing in diameter from the proximal end towards the distal end, allowing for a user's mouth to fit comfortably therearound. In some embodiments, the mouthpiece is rectangular or oval shaped, having a wider surface to distribute vapor evenly.

Referring now to FIG. 2, there is shown a semi-cross-sectional view of the portable alcohol vaporizer, taken along lines 2-2 of FIG. 1. The handle body 1200 houses a rechargeable battery 1500 configured to provide electrical power to a heating element 2100 located in the tip 1400. The battery 1500 is operatively connected to a printed circuit board assembly (PCBA) 2200, which controls the vaporization process. The PCBA 2200 manages the flow of power from the battery 1500 to the heating element 2100 and communicates with various user interface elements.

In the shown embodiment, the tip 1400 contains the liquid reservoir 1420, which is in fluid communication with the heating element 2100 via a wick 2300. The wick 2300 transfers the concentrated alcohol compound 2000 from the reservoir 1420 to the heating element 2100, ensuring a consistent supply of alcohol for vaporization. In one embodiment, a pump 2400 is positioned between the reservoir 1420 and the wick 2300 to control the flow rate of the alcohol compound 2000 to the heating element 2100, providing precise delivery for optimal vaporization.

In one embodiment, the heating element 2100 is disposed within a vaporization chamber 2500, where the concentrated alcohol compound 2000 is heated to produce alcohol vapor. In one embodiment, the heating element 2100 is a resistive heating coil configured to generate heat by passing an electric current through a conductive material. In alternative embodiments, the heating element comprises ceramic heating plates, which provide even heat distribution and maintain consistent temperatures for vaporizing the alcohol compound 2000, and/or an infrared heating elements, which generate heat through electromagnetic radiation. The vaporization chamber 2500 is thermally insulated to ensure efficient heating while minimizing heat transfer to the surrounding components. The vaporized alcohol is transported from the vaporization chamber 2500 through a vapor tube 2600 that connects to the mouthpiece 1600. The vapor tube 2600 is configured to minimize condensation and ensure efficient delivery of the vapor to the user. The mouthpiece 1600 is designed to be ergonomic and comfortable for the user, providing a clear and direct pathway for inhalation.

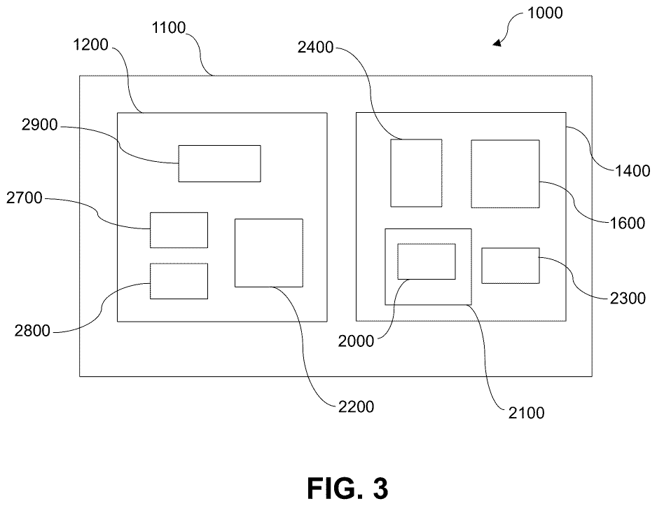

Referring now to FIG. 3, there is shown a block diagram of the electronic components of the portable alcohol vaporizer 1000. In the shown embodiment, the battery 1500 is operatively connected to a printed circuit board assembly (PCBA) 2200, which regulates the power distribution to various components of the device. The PCBA 2200 is programmed to control the operation of the heating element 2100, manage user inputs, and monitor device safety parameters. The heating element 2100, which is responsible for vaporizing the alcohol compound 2000, receives power directly from the PCBA 2200. The PCBA 2200 ensures that the heating element 2100 operates within a safe temperature range, preventing overheating and ensuring consistent vaporization.

In the shown embodiment, the PCBA 2200 is configured to interact with user controls. These include a button 2700 for manual activation and an optional sensor 2800 positioned near the mouthpiece 1600. The sensor 2800 detects the presence of the user's mouth and automatically activates the heating element 2100, providing a hands-free operation option.

The liquid reservoir 1420 is connected to the heating element 2100 via a wick 2300 or, in some embodiments, a pump 2400. The pump 2400, if included, is electronically controlled by the PCBA 2200 to regulate the flow rate of the alcohol compound 2000 to the heating element 2100. The PCBA 2200 is also connected to user interface elements. These include a button 2700 for manual activation of the device and an optional sensor 2800 near the mouthpiece 1600 that detects the presence of a user's mouth. The PCBA 2200 processes input from the button 2700 or sensor 2800 and activates the heating element 2100 accordingly.

In some embodiments, safety features are integrated into the PCBA 2200 to protect the user and the device. For example, the PCBA 2200 includes circuitry to prevent the heating element 2100 from operating when the liquid reservoir 1420 is empty. Additionally, the PCBA 2200 can include time-controlled dispensing functionality to limit the duration of vapor release per activation, reducing the risk of excessive alcohol consumption. Feedback to the user is provided through indicator lights 2900, which are operatively connected to the PCBA 2200. These lights indicate the status of the device, such as battery charge level, operational readiness, and liquid reservoir capacity. The lights 2900 are disposed on the housing.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A portable alcohol vaporizer, comprising:
a housing including a handle body and a tip;
a liquid reservoir configured to hold a concentrated alcohol compound;
a heating element disposed in a chamber, the chamber in fluid communication with the liquid reservoir, wherein the heating element is configured to vaporize the concentrated alcohol compound into vaporized alcohol;
a vapor tube in fluid communication with the chamber and a mouthpiece, wherein the vapor tube is configured to deliver the vaporized alcohol to the mouthpiece;
a battery disposed within the handle body configured to power the heating element;
a printed circuit board assembly (PCBA) configured to control the vaporization process and operatively connected to the battery and the heating element.

2. The portable alcohol vaporizer of claim 1, wherein the heating element is a heating coil.

3. The portable alcohol vaporizer of claim 1, wherein the handle body includes a charging port for recharging the battery.

4. The portable alcohol vaporizer of claim 1, wherein the tip is removably connected to the handle body via a fastener.

5. The portable alcohol vaporizer of claim 4, wherein the fastener comprises mating threaded fasteners disposed on the handle body and the tip.

6. The portable alcohol vaporizer of claim 5, wherein tip and handle body form a pen shaped housing when secured to each to each other via the fastener.

7. The portable alcohol vaporizer of claim 1, further comprising a wick disposed between the liquid reservoir and the heating element, the wick configured to control the transfer of the alcohol compound for vaporization.

8. The portable alcohol vaporizer of claim 1, further comprising a pump having a valve configured to control the flow of the concentrated alcohol compound to the heating element.

9. The portable alcohol vaporizer of claim 1, wherein the pump is configured to move a metered volume of the concentrated alcohol compound to the heating element.

10. The portable alcohol vaporizer of claim 1, further comprising a sensor configured to detect the presence of a user's mouth on the mouthpiece and actuate the heating element to vaporize the concentrated alcohol compound in response.

11. The portable alcohol vaporizer of claim 1, wherein the user interface includes a button operatively connected to the PCBA, the button configured for selective activation of the heating element.

12. The portable alcohol vaporizer of claim 1, further comprising a time-controlled dispensing mechanism configured to limit the duration of alcohol vapor release per activation.

13. The portable alcohol vaporizer of claim 1, wherein the liquid reservoir is refillable with a concentrated alcohol compound via a refill port.

14. The portable alcohol vaporizer of claim 1, wherein the liquid reservoir is a cartridge that is interchangeable with a second similar liquid reservoir cartridge.

15. The portable alcohol vaporizer of claim 1, further comprising an indicator light or display configured to provide information regarding battery status, operational readiness, or remaining liquid level.
